(19) United States
(12) Patent Application Publication
(10) Pub. No.: US 2019/0328373 A1
(43) Pub. Date: Oct. 31, 2019

(54) CATHETER-SHAPED SAMPLING DEVICE AND METHODS THEREOF

(71) Applicants: Xibo Wei, Hayward, CA (US); Xiyi Wei, Ningbo (CN)

(72) Inventors: Xibo Wei, Hayward, CA (US); Xiyi Wei, Ningbo (CN)

(21) Appl. No.: 16/507,026

(22) Filed: Jul. 10, 2019

(51) Int. Cl. A61B 10/04; A61M 25/01

(52) U.S. Cl. CPC A61B 10/04; A61B 10/0283; A61M 25/01

(57) ABSTRACT

The present invention provides a device that can move inside a biological conduit such as an intestinal tract for sampling the content therein, and a process thereof. The device includes a flexible tube, a proximal handling system connected to a proximal end of the tube; and one or more sampling channels within the tube. Discrete hairs and/or continuous belt in the sampling channels are designed to collect the content within the conduit. The hairs and belt loaded with the sample may be used to establish a chemical, biochemical and microbiological spectrum along the length of the intestinal tract.

### FIELD OF THE INVENTION

The present invention generally relates to a catheter-shaped sampling device capable of discontinuously and/or continuously sampling the content in a biological conduit, and a method thereof, The conduit may be a biological conduit such as human or animal intestinal tract or gut, but it may also be an industrial conduit. Embodiments of this invention can advance the fields of intestinal endocrinology, endoscopy, and microbiology by extending the sampling device into sections of the intestinal tract and obtaining biochemical and microbe samples in conjunction with a sample gathering and handling system, data acquisition, and data analysis. Although the invention will be illustrated, explained and exemplified with a sampling device for the intestinal tract, it should be appreciated that the present invention can serve clinical diagnostic, treatment and research purposes in other fields, for example, biological conduit including gastrointestinal tract, respiratory tract, and the like.

### BACKGROUND OF THE INVENTION

Many times, the content in a biological conduit needs to be sampled and analyzed. Take human or animal intestinal tract or gut as an example. Small intestine (AKA small bowel) is the part of the gastrointestinal tract between the stomach and the large intestine. The average length of the small intestine in a living person is about 6 meters. The small intestine does not only digest food and absorb nutrients and minerals, but also support the body's immune system. Jejunum is the midsection of the small intestine, connecting the duodenum to the ileum. It is about 2.5 m long, and contains the plicae circulares, and villi that increase its surface area. In the jejunum, digestion products such as sugars, amino acids, and fatty acids are absorbed into the bloodstream. The ileum is the final section of the small intestine, and is about 3 m long. The ileum absorbs mainly vitamin B12 and bile acids, as well as any other remaining nutrients. The ileum joins to the cecum of the large intestine at the ileocecal junction. Peyer's patches located within the ileum are an important part of the digestive tract's local immune system. These patches are part of the lymphatic system, and provide a site for antigens from potentially harmful bacteria or other microorganisms in the digestive tract to be sampled, and subsequently presented to the immune system The jejunum and ileum are suspended in the abdominal cavity by mesentery. The mesentery is part of the peritoneum. Arteries, veins, lymph vessels and nerves travel within the mesentery.

Large intestine (AKA large bowel) includes many sections along its 1.5-meter length, such as cecum and appendix, ascending colon, transverse colon, descending colon, sigmoid colon, rectum, and anal canal. Lymphatic drainage from the ascending colon and proximal two-thirds of the transverse colon is to the colic lymph nodes and the superior mesenteric lymph nodes, which drain into the cisterna chyli. The lymph from the distal one-third of the transverse colon, the descending colon, the sigmoid colon, and the upper rectum drain into the inferior mesenteric and colic lymph nodes. The lower rectum to the anal canal above the pectinate line drain to the internal iliac nodes. The anal canal below the pectinate line drains into the superficial inguinal nodes.

Moreover, the immunological function of the intestinal tract is also associated with the complex community of microorganisms called gut flora or gut microbiota. For example, the large intestine houses over 700 species of bacteria that perform a variety of functions, as well as fungi, protozoa, and archaea. The amount of microbes in a human distal gut is in the vicinity of 100 trillion. In humans, the gut flora is established at one to two years after birth, and by that time, the intestinal epithelium and the intestinal mucosal barrier that it secretes have co-developed in a way that is tolerant to, and even supportive of, the gut flora and that also provides a barrier to pathogenic organisms. Some human gut microorganisms benefit the host by fermenting dietary fiber into short-chain fatty acids (SCFAs), such as acetic acid and butyric acid. Intestinal bacteria also play a role in synthesizing vitamin B and vitamin K as well as metabolizing bile acids, sterols, and xenobiotics. The systemic importance of the SCFAs and other compounds they produce are like hormones, and the gut flora itself appears to function like an endocrine organ. Dysregulation of the gut flora has been correlated with a host of inflammatory and autoimmune conditions.

People tend to underestimate the importance of gut flora, which plays many key roles. The gut flora community defends against pathogens by fully colonizing the space, making use of all available nutrients, and by secreting compounds that kill or inhibit unwelcome organisms that would compete for nutrients with it. It develops and maintains the intestinal epithelium and inducing antibody production. It helps to metabolize otherwise indigestible compounds in food The gut flora may even train and develop the immune system.

Surprisingly, recent study shows that biochemical signaling can take place between the gastrointestinal tract and the central nervous system, via the so-called “gut-brain axis”. The gut-brain axis includes the central nervous system, neuroendocrine and neuroimmune systems including the hypothalamic-pituitary-adrenal axis (HPA axis), sympathetic and parasympathetic arms of the autonomic nervous system including the enteric nervous system and the vagus nerve, and the gut microbiota. As a bidirectional neurohumoral communication system, the gut-brain axis is important for maintaining homeostasis and is regulated through the central and enteric nervous systems and the neural, endocrine, immune, and metabolic pathways. The gut flora can produce a range of neuroactive molecules, such as acetylcholine, catecholamines, γ-aminobutyric acid, histamine, melatonin, and serotonin, which is essential for regulating peristalsis and sensation in the gut. Changes in the composition of the gut flora due to diet, drugs, or disease correlate with changes in levels of circulating cytokines, some of which can affect brain function. Likewise, chronic or acutely stressful situations activate the hypothalamic-pituitary-adrenal axis, causing changes in the gut flora and intestinal epithelium, and possibly having systemic effects. Additionally, the cholinergic anti-inflammatory pathway, signaling through the vagus nerve, affects the gut epithelium and flora. Hunger and satiety are also integrated in the brain. There may be a relationship between the gut flora and anxiety disorders and mood disorders including depression, schizophrenia, autism, Parkinson's disease, and obsessive-compulsive disorder.

That being said, it is critically important to sample and study the content in an intestinal tract. However, the human gut is largely unexplored, except for post mortem autopsies, which do not reflect much of the most important living dynamic phenomena and conditions. Many diseases with origin in human gut are of unknown causes, so only symptoms are treated. For example, the remote and inaccessible 15′ sections of intestines, beyond the 6 feet up and 6 feet down as viewed by endoscopy/colonoscopy instruments, remain unexplored in live humans. Even these upper and lower extremities that are viewed by camera can only be treated for visible damage, such as polyps or ulcers. A camera pill may be swallowed, and pictures are taken throughout the intestinal tract, but the information gleaned is largely qualitative. As such, quantitative in-vivo data and measurements are generally not available.

There is hence a serious need to analyze the biochemical, biological, physiological, and bioengineering processes taking place within the entire human intestinal tract, as precisely as in other anatomical systems (e.g. DNA, and microbiology). Advantageously, the present invention provides a sampling device capable of discontinuously and/or continuously sampling the content in human or animal intestinal tract, and a method thereof. The present invention enables an easy and convenient operation for collection and preservation of intestinal bio/chemical profile for external analysis, and measurements of many in vivo conditions. The invention can support medical professionals' decisions-making such as construction of mathematical, physiological, biochemical, and other engineering models; delineation of causes of diseases originating within the gut; and prescribing diets or medications.

### SUMMARY OF THE INVENTION

One aspect of the present invention provides a catheter-shaped sampling device that can enter into a conduit, and can move along the conduit for sampling a content contained therein. The catheter-shaped sampling device includes:
a flexible tube insertable into a conduit for sampling content contained therein;
a proximal handling system (member) connected to a proximal end of the flexible tube;
one or more sampling channels within the flexible tube each having a distal opening at a distal end of the flexible tube; and
a discontinuous sample collector in one of said sampling channels that collects the content within the biological conduit.

In a representative embodiment, the catheter-shaped sampling device includes a continuous sample collector in one of the sampling channels, in addition to or as an alternative of, the discontinuous sample collector as described above.

Another aspect of the invention provides a process for sampling content in a conduit using the catheter-shaped sampling device as described above. The process includes the steps of inserting the flexible tube into the conduit; moving the flexible tube along the conduit; and collecting the content of the conduit at a targeted location with a discontinuous sample collector and/or a continuous sample collector.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 schematically shows a catheter-shaped sampling device in accordance with an exemplary embodiment of the present invention.

FIG. 2 schematically demonstrates a sampling device built like a colonoscope for inserting into the intestinal tract through anus in accordance with an exemplary embodiment of the present invention.

FIG. 3 schematically illustrates a sampling device built like an esophagogastroduodenoscope (EGD) with two additional balloons for facilitating its movement inside the intestinal tract in accordance with an exemplary embodiment of the present invention.

FIG. 4 shows a discontinuous catheter-shaped sample collector with sampling hairs in accordance with an exemplary embodiment of the present invention.

FIG. 5 illustrates a sampling operation using the discontinuous catheter-shaped sample collector with sampling hairs in accordance with an exemplary embodiment of the present invention.

FIG. 6 shows a continuous catheter-shaped sample collector with sampling belts in accordance with an exemplary embodiment of the present invention.

FIG. 7 is a sectional view that schematically depicts a design of the continuous sample collector assembly including two belt sources and a sample depository in accordance with an exemplary embodiment of the present invention.

FIG. 8 schematically illustrates the structure of a convoy belt with length markers in accordance with an exemplary embodiment of the present invention.

FIG. 9 is a the flow chart of a process for sampling content in a conduit using the catheter-shaped sampling device in accordance with an exemplary embodiment of the present invention.

FIG. 10 shows two convoy belts sandwiching intestinal sample being cut into many small pieces for detailed analysis in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

The term “conduit” is intended to include a biological conduit, an industrial conduit, or any combination thereof. Examples of the biological conduit include, but are not limited to, human or animal intestinal tract or gut, gastrointestinal tract, buccal cavity, pharynx, esophagus, stomach, small intestine, duodenum, jejunum, ileum, large intestine, cecum, colon, rectum, anal canal, respiratory tract, upper respiratory tract, lower respiratory tract, nasal cavity, paranasal sinuses, pharynx, nasopharynx, oropharynx, laryngopharynx, larynx, trachea, bronchi (primary, secondary and tertiary), bronchioles (including terminal and respiratory), lungs (including alveoli), ear canal, vagina, cervix, uterus or womb, Fallopian tubes, ovaries, urinary tract, kidney, renal pelvis, ureter, urinary bladder, and urethra etc. Examples of the industrial conduit include, but are not limited to, various tubular structures in an industrial apparatus, equipment, a product, a machine, a production line, and the like.

The term “content’ as used herein may include human or animal gastrointestinal fluid, intestinal secretions, solid or semisolid, substances, slurry, feces, and stool etc.

In the following description, the invention will be illustrated and described using intestinal tract as a representative example. However, it should be appreciated that the invention may be applied to any other conduit structures for sampling the content therein, mutatis mutandis.

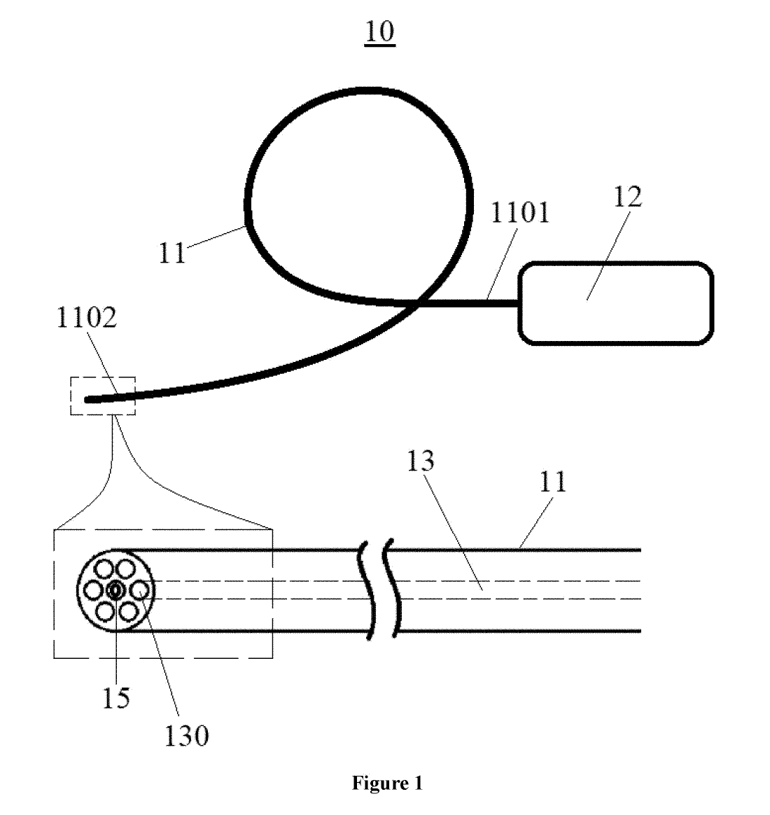

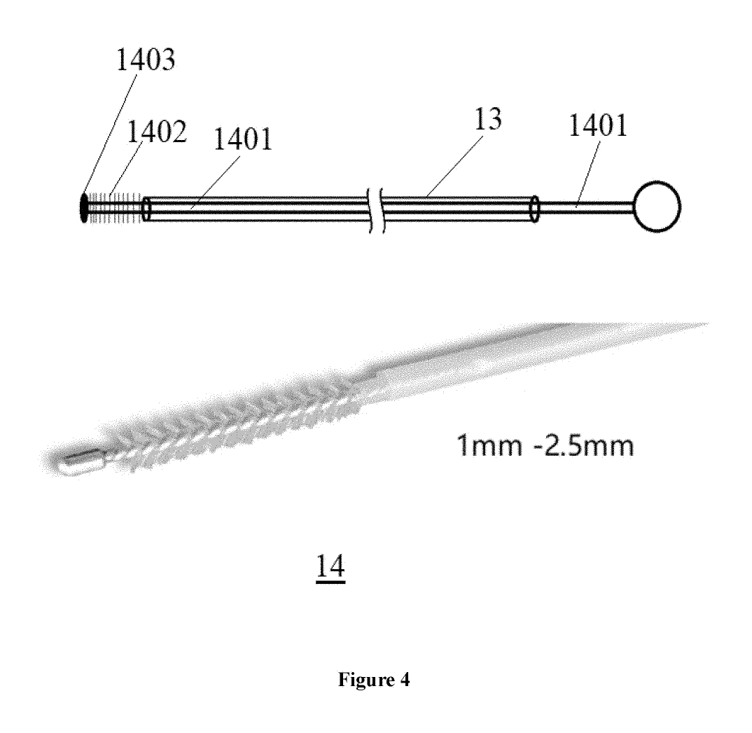

Referring now to FIG. 1, a catheter-shaped sampling device 10 includes a flexible tube 11 that is insertable into a conduit such as a biological conduit for sampling the content contained therein. Tube 11 may be made of an inert material that does not cause irritation, such as polypropylene. The length of tube 11 may be about 5 meters. A proximal handling system (or member) 12 is connected to a proximal end 1101 of the flexible tube 11, and will be located outside the conduit such as the intestinal tract during its usage. Each of the one or more sampling channels 13 within the flexible tube 11 has a distal opening 130 at a distal end 1102 of the flexible tube 11. A discontinuous sample collector 14 (as shown in FIG. 4) may be located in one of sampling channels 13 that collects the content within the conduit. Other known components 15 such as a camera and a lighting device may be assembled at the distal end 1102 of the flexible tube 11. Optionally, such sampling device design may include other components 15 such as a light delivery system to illuminate the intestinal tract. The light source is normally outside the patient's body and the light is typically directed via an optical fiber. A lens system may transmit the image from the objective lens to the viewer. One or more additional channels may be used to insert medical instruments or manipulators into the intestinal tract.

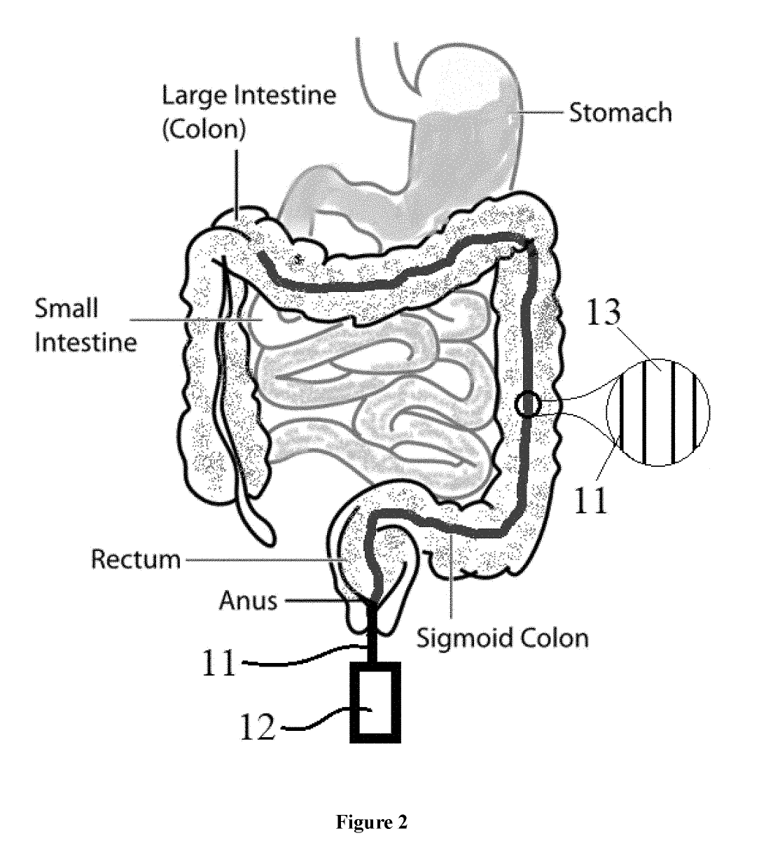

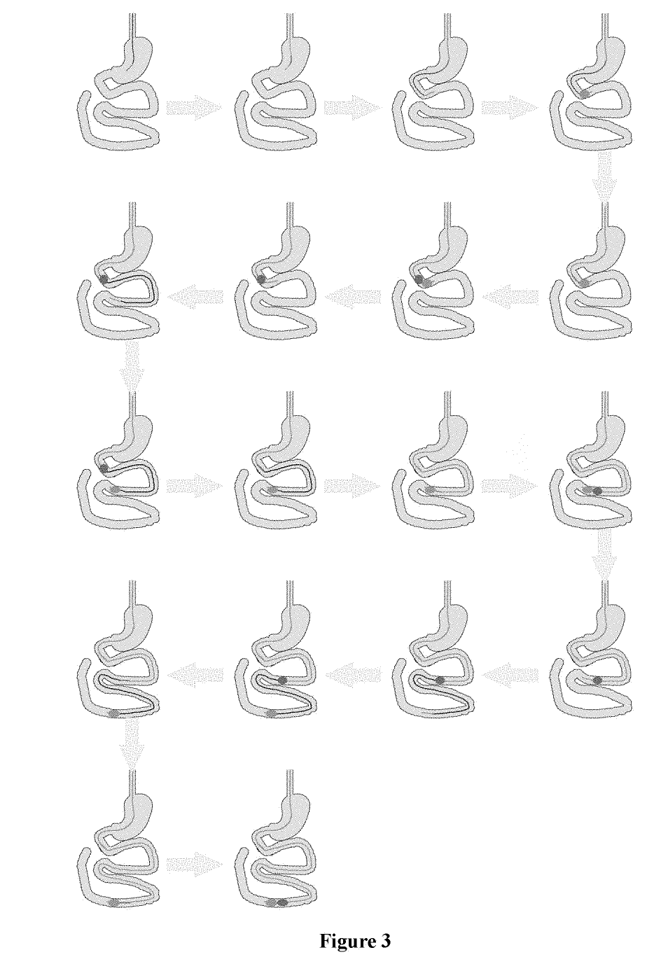

The catheter-shaped sampling device 10 of the invention may be built like a probe, a gastric feeding tube, a duodenal feeding tube, or a gastrostomy, among others. For example, the catheter-shaped sampling device 10 may be built like, and used like, a colonoscope, for inserting into the intestinal tract through a patient's anus as shown in FIG. 2. It may also be built like, and used like, an esophagogastroduodenoscope (EGD) with two additional balloons for facilitating its movement inside the intestinal tract, after it is inserted through a patient's mouth, as shown in FIG. 3.

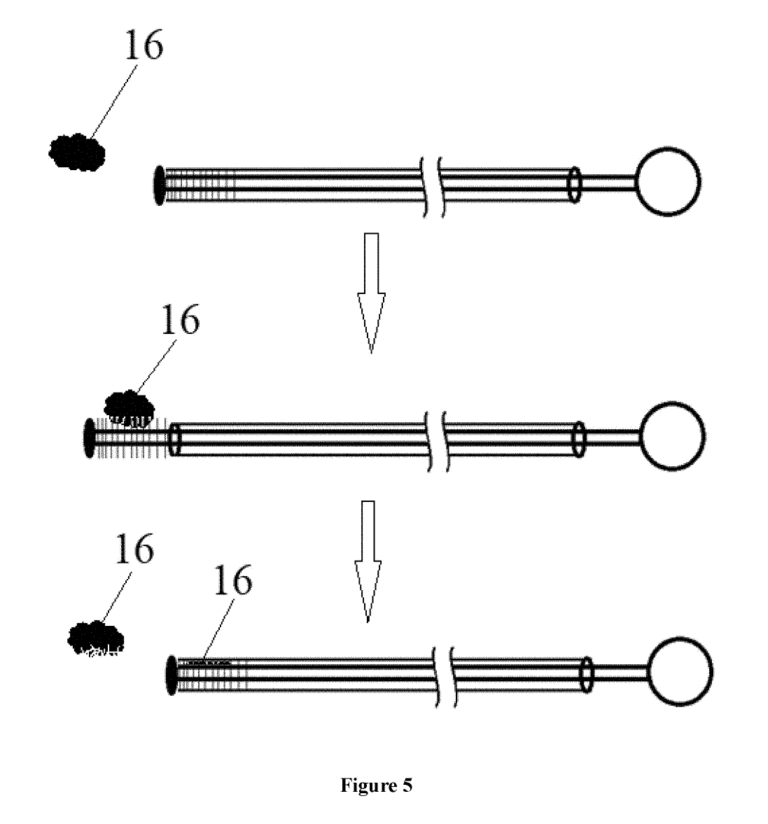

Referring to FIG. 4, the discontinuous sample collector 14 may include a sampling wire 1401 within the sampling channel 13. Sampling hairs 1402 are preferably extended from a distal and terminal portion of the sampling wire 1401. A protective cap 1403 is connected to the distal tip of the sampling wire 1401. Cap 1403 is slightly bigger than the distal opening 130 such that it can removably cover and seal the distal opening 130 to prevent the sampling hairs 1402 and/or samples 16 adhered to (or taken by) the sampling hairs 1402 within the sampling channel 13 from contamination by foreign substances. Cap 1403 may be made of a material, such as cellulose acetate phthalate, glyceryl stearates, paraffin, or epoxy compounds. In a sampling operation as shown in FIG. 5, sampling hairs 1402 can be pushed out from the sampling channel 13 by pushing the wire 1401 forward (toward distal direction) until hairs 1402 reach sample 16, and adhere at least a portion of sample 16. Then, hairs 1402 are pulled back into the sampling channel 13 with the adhered sample 16, by pulling the wire 1401 backward (toward proximal direction), until the cap 1403 covers and seals the distal opening 130.

As an alternative to, or in addition to, the discontinuous sample collector 14, a continuous sample collector 24 may be included in one of sampling channels 13. In various embodiments, continuous sample collector 24 generally employs a rolling belt design to collect the intestinal content.

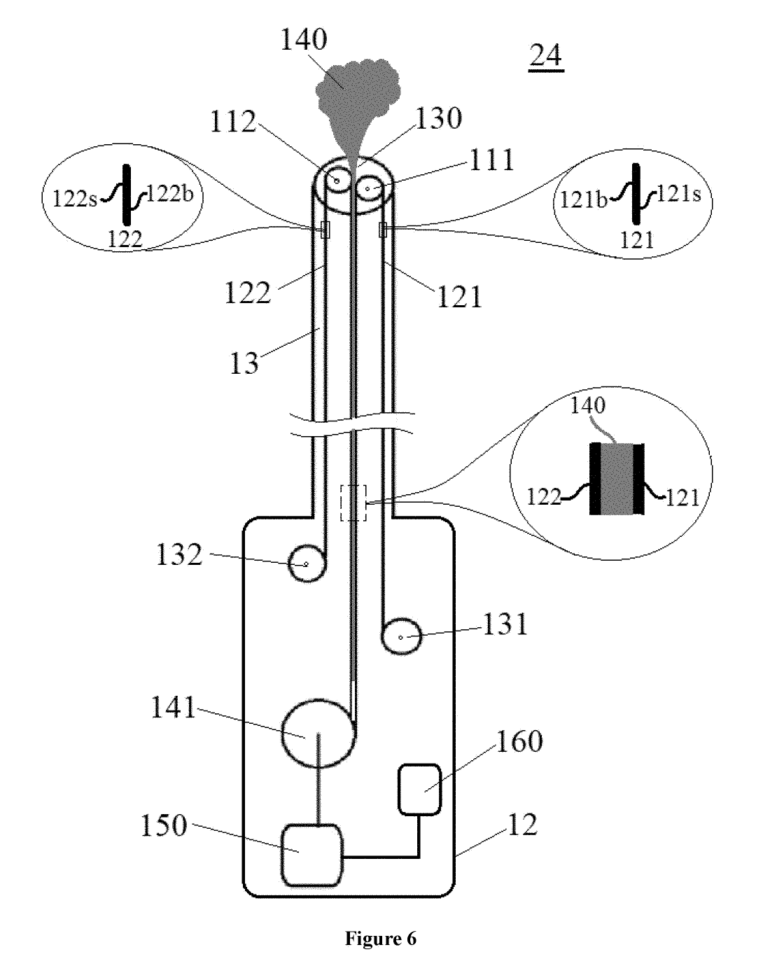

In an embodiment as shown in FIG. 6, a continuous sample collector 24 includes
a first cylindrical roller 111;
a first convoy belt 121 having a first sampling surface 121s and a first back surface 121b,
a first belt source 131 for providing the first convoy belt 121 which is blank or clean;
a second cylindrical roller 112;
a second convoy belt 122 having a second sampling surface 122s and a second back surface 122b, and
a second belt source 132 for providing the second convoy belt 122 which is blank.

Rollers 111 and 112 work together like twin roller strip caster, in which the two rollers have parallel axes and are spaced apart from each other by a distance corresponding to the desired thickness of the cast strip. Content 140, like ingot steel, is fed or supplied to the gap between two oppositely-rotating casting rolls 111 and 112, forming a sheet-like sample that can be rolled into sample depository 141.

The first convoy belt 121 is so configured that, in a sampling operation, it extends from the first belt source 131 to the first cylindrical roller 111. Then, it contacts the roller 111 with its first back surface 121b. While belt 121 is winding around the roller 111, the first sampling surface 121s is exposed to the content 140 in the conduit. Sampling surface 121s is loaded (or absorbed) with a portion of the content 140 by adhesion. At last, convoy belt 121 carries the loaded content 140 to a sample depository 141. Sample depository 141 stores the samples.

The second convoy belt 122 works in a manner similar to belt 121. Belt 122 operates in a symmetrical manner with belt 121. It is configured to extend from the second belt source 132 to the second cylindrical roller 112, to contact the roller 112 with the second back surface 122b, to wind around the second cylindrical roller 112, to expose the second sampling surface 122s to the content 140 in the conduit, to load the content 140 on the second sampling surface 122s, and to carry the loaded content 140 to the sample depository 141.

Therefore, the contents 140 on surfaces 121s and 122s will meet each other and be properly squeezed together when belts 121 & 122 pass the gap between two rollers 111 & 112. The gap is configured to be narrower than the thickness of combined belts 121 & 122 with content 140, so that content 140 is completely squeezed (but not over squeezed it to cause content oozing out) between the two surfaces 121s and 122s, and completely loaded on, or sandwiched/encapsulated between, both surfaces. Then belts 121 & 122 both pass through the gap between rollers 111 and 112, and carry the loaded content 140 to the sample depository 141. Such a “sandwich” or “encapsulation” configuration helps to preserve the sample 140's in-vivo environmental integrity, and to prevent cross contamination between different samples on the same belt. At last, the two convoy belts 121 & 122 loaded with the combined contents 140 are pulled at a same, speed toward the sample depository 141.

As shown in FIG. 6, the two cylindrical rollers (111, 112) are in parallel with each other, and have a gap therebetween. Two convoy belts (121, 122) pass through the gap after they are loaded with their respective content 140 on their sampling surfaces (121s, 122s). Both convoy belts (121, 122) will carry their loaded contents 140 to the sample depository 141. The gap is so configured that the contents 140 loaded on the two convoy belts (121, 122) are combined, and the combined contents 140 are sandwiched between the two convoy belts (121, 122), forming a stacked structure (121-140-122), as shown in the enlarged view in FIG. 6.

A manual or powered collector driver 150 may be configured to pull the two convoy belts (121, 122) loaded with the combined contents 140 at a same speed toward the sample depository 141. The sampling device of the invention may further include a controller 160 to manage the operation of the device. If necessary, a vacuum system (not shown) may be employed to suck the intestinal content into channel 13 through its opening 1301.

The convoy belts (121, 122) used in the present invention is preferably motor-driven, thin, and sterile ribbon-belt. Samples from different sections of the intestinal tract can be collected into different locations of the convoy belts.

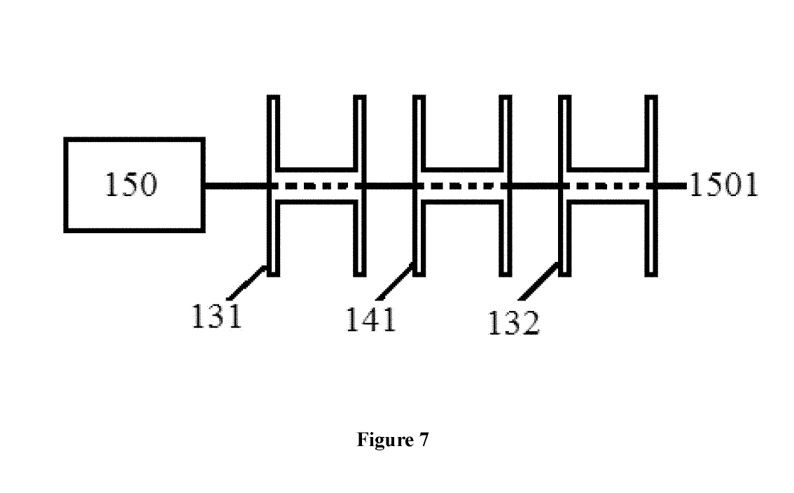

In a specific embodiment as shown in FIG. 7, a motor 150 rotates a shaft 1501 either clockwise or anticlockwise First belt source 131 comprises a roll/reel/spool of the first convoy belt 121 (fresh, blank and unloaded) around a roller, First belt source may be a roll/reel/spool 131 that is installed around shaft 1501, but it does not engage with the shaft 1501, and is not drivable by the shaft 1501. Therefore, roll/reel/spool 131 can rotate freely around shaft 1501. Second belt source 132 comprises a roll/reel/spool of the first convoy belt 122 (fresh, blank and unloaded) around a roller. Second belt source is a roll/reel/spool 132 that is also installed around shaft 1501, but it does not engage with the shaft 1501 either, and is not directly drivable by the shaft 1501 either. Therefore, roll/reel/spool 132 can rotate freely around shaft 1501. The sample depository may be a collection roll/reel/spool 141 that is installed between 131 and 132 around the shaft 1501. Collection roll/reel/spool 141 does engage with the shaft 1501, and is directly drivable by the shaft 1501. Therefore, motor 150 can directly rotate collection roll/reel/spool 141, and indirectly rotate rolls/reels/spools 131 and 132 through belts 121 & 122.

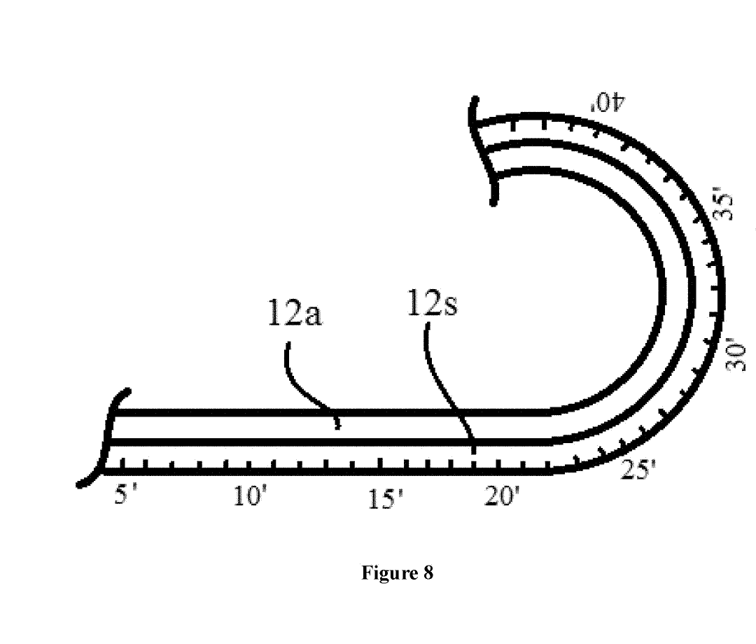

In various embodiments, the convoy belt 121/122 of the invention may include two layers joined together, a substrate layer having a back surface as described above, and an adsorbent layer having a sampling surface as described above. As shown in FIG. 8, substrate layer 12s may be a strip of fabric made of multiple plies of a material such as polyester (PES), Nylon or polyamide (PA). The substrate layer 12s functions to increase the tensile strength of the convoy belt 121/122 used in the present invention, and to survive the force that pulls them toward the sample depository 141. The adsorbent layer 12a is preferably continuous, homogenous, flat, smooth and free of any indentations and pockets, and may comprise a material selected form sodium carboxymethylcellulose, polyacrylamide, polyacrylonitrile and acrylic acid polymers, cross-linked acrylic, Karaya gum and polysaccharides Adsorbent layer 12a may be cured from a monomer solution applied to or impregnated within the substrate layer 12s.

In a preferred embodiment as shown in FIG. 8, convoy belts 121 and/or 122 as described above may be marked with length indicators. The sampling device may be configured to, or programmed to, correlate (A) the specific intestinal sample 140 loaded on the belt 121/122 at a length indicator (e.g. 35 inches) to (B) the location in the intestinal tract where that sample is collected (e.g. 6 feet away from the mouth). With the correlation, a user can establish (1) the spectrum of biochemical products as a function of the intestinal tract length; (2) the spectrum of biochemical reactions as a function of the intestinal tract length; (3) the spectrum of microbes as a function of the intestinal tract length; (4) the spectrum of byproducts of all microbes as a function of the intestinal tract length, and (5) the spectrum of aerobic/anaerobic distribution and associated conditions as a function of the intestinal tract length.

In an embodiment, convoy belt 122 as described above may be a simple covering belt with no adsorbent layer 12a, and such a covering belt may simply be used to seal, cover, or encapsulate belt 121 that is loaded with intestinal sample 140.

In some embodiments, belt 121 may function as a sample wipe or a sample trap. It may be made of paper, cotton cloth or porous PTFE (Teflon), for example, an open weave fiberglass fabric coated with a layer of PTFE.

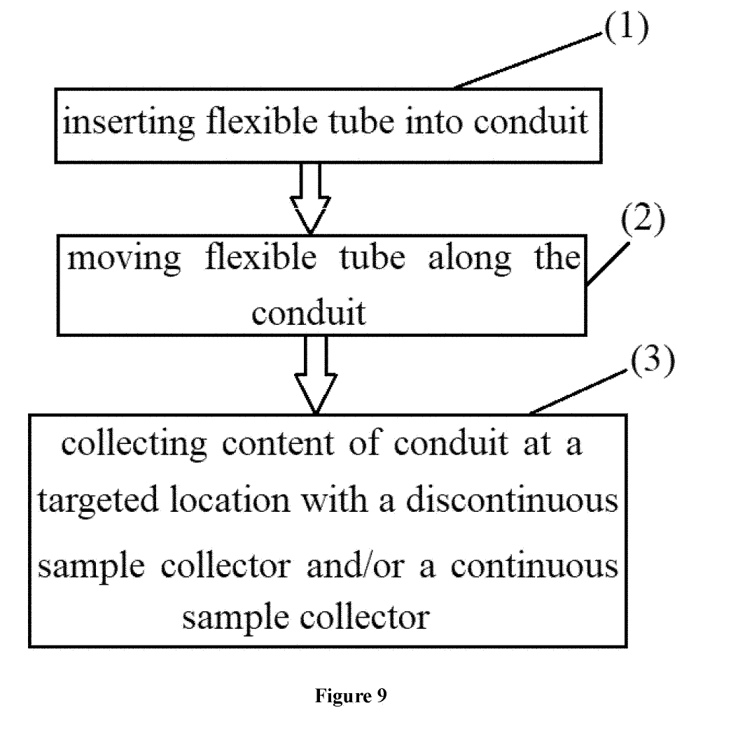

The present invention also provides a process for sampling content in a conduit using the catheter-shaped sampling device. The position of the sampling device may be monitored with ultrasound, X-ray, magnetic resonance imaging (MRI), or other means, and when the device reaches a predetermined position/section of the intestinal tract, the device may start collecting samples along the pathway in the intestinal tract. As shown in FIG. 9, the process includes
(1) inserting the flexible tube 11 into the conduit;
(2) moving the flexible tube 11 along the conduit; and
(3) collecting the content of the conduit at a targeted location with a discontinuous sample collector 14 and/or a continuous sample collector 24.

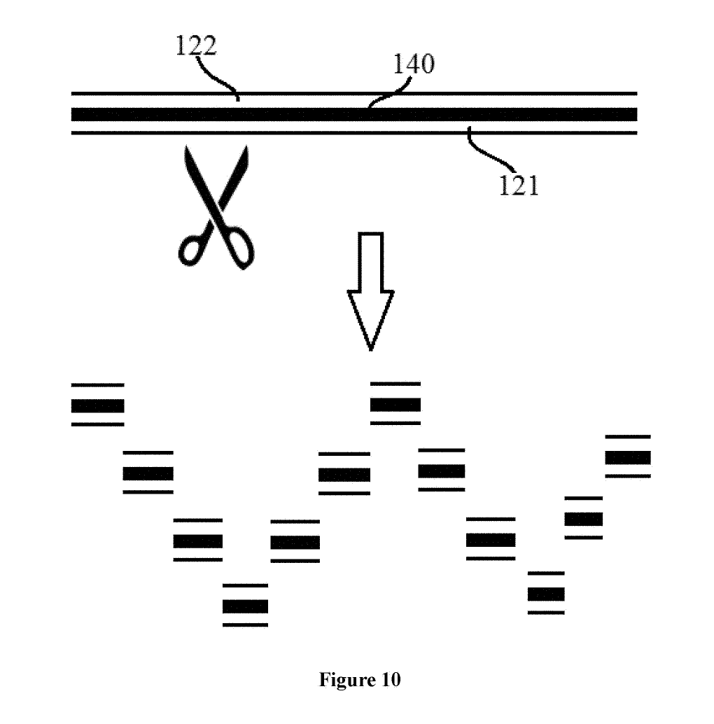

The sample depository 141 with the collected sample 140 is subsequently sent or handed over to a medical institution or laboratory, where the sample is analyzed. The loaded belt 121 or belts 121/122 may mirror the content of at least a segment of the intestinal tract along its length, and can be cut into pieces to establish the spectrum of intestinal content in that segment. Referring to FIG. 10, two convoy belts 121 & 122 loaded with the combined contents 140 are cut into pieces for analyzing the sample content 140, as a function of intestinal tract length. With a sufficient number of measurements, a user can conduct sample chemical analysis, microbe strains/colonies identification and characterization along the entire intestinal tract.

The individual hairs 1402 and the pieces in FIG. 10 are useful for establishing the following medical information associated with heathy or unhealthy individuals (1) the spectrum of biochemical products as a function of the intestinal tract length, e.g. as a result of any specific diet, (2) the spectrum of biochemical reactions as a function of the intestinal tract length; (3) the spectrum of microbes as a function of the intestinal tract length; (4) the spectrum of byproducts of all microbes as a function of the intestinal tract length; (5) the spectrum of aerobic/anaerobic distribution and associated conditions as a function of the intestinal tract length; (6) the interaction of a) the normal biochemical reactions, b) the microbes, and c) the microbe byproducts that causes several major diseases, as a function of the intestinal tract length; (7) data necessary to identify and characterize physiological, biochemical or other bio-engineering processes; (8) the microbial flora distribution for the intestinal tract as a function of the intestinal tract length; and (9) the ratios of solids, liquids, and gases, as well as, their compositions, temperature, partial pressures, and other variables, as a function of the intestinal tract length.

In the foregoing specification, embodiments of the present invention have been described with reference to numerous specific details that may vary from implementation to implementation. The specification and drawings are, accordingly, to be regarded in an illustrative rather than a restrictive sense The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A catheter-shaped sampling device 10 comprising:
a flexible tube 11 that is insertable into a conduit such as a biological conduit for sampling content contained therein;
a proximal handling system (member) 12 connected to a proximal end 1101 of the flexible tube 11;
one or more sampling channels 13 within the flexible tube 11 each having a distal opening 130 at a distal end 1102 of the flexible tube 11; and
a discontinuous sample collector 14 in one of said sampling channels 13 that collects the content within the conduit.

2. The catheter-shaped sampling device according to claim 1, further comprising other components 15 such as a camera and a lighting device at the distal end 1102 of the flexible tube 11.

3. The catheter-shaped sampling device according to claim 1, wherein the discontinuous sample collector 14 includes
a sampling wire 1401 within the sampling channel 13,
sampling hairs 1402 extended from a distal and terminal portion of the sampling wire 1401, and
a protective cap 1403 connected to a distal tip of the sampling wire 1401;
wherein the protective cap 1403 is slightly bigger than the distal opening 130 such that the protective cap 1403 can cover and seal the distal opening 130 to prevent the sampling hairs 1402 and/or samples 16 adhered to the sampling hairs 1402 within the sampling channel 13 from contamination by foreign substances.

4. The catheter-shaped sampling device according to claim 3, wherein sampling hairs 1402 can be pushed out from the sampling channel 13, adhered to some of sample 16, and pulled back into the sampling channel 13 with the adhered sample 16.

5. The catheter-shaped sampling device according to claim 1, further comprising a continuous sample collector 24 in one of said sampling channels 13.

6. The catheter-shaped sampling device according to claim 5, wherein the continuous sample collector 24 comprises
a first cylindrical roller 111;
a first convoy belt 121 having a first sampling surface 121s and a first back surface 121b;
a first belt source 131 for providing the first convoy belt 121 which is blank;
a sample depository 141, wherein the first convoy belt 121 is configured to extend from the first belt source 131 to the first cylindrical roller 111, to contact the roller 111 with the first back surface 121b, to wind around the roller 111, to expose the first sampling surface 121s to the content 140 in the conduit, to load a portion of the content 140 on the first sampling surface 121s, and to carry the loaded content 140 to the sample depository 141;
a second cylindrical roller 112;
a second convoy belt 122 having a second sampling surface 122s and a second back surface 122b; and
a second belt source 132 for providing the second convoy belt 122 which is blank;
wherein the second convoy belt 122 is configured to extend from the second belt source 132 to the second cylindrical roller 112, to contact the roller 112 with the second back surface 122b, to wind around the second cylindrical roller 112, to expose the second sampling surface 122s to the content 140 in the conduit, to load the content 140 on the second sampling surface 122s, and to carry the loaded content 140 to the sample depository 141;
wherein the two cylindrical rollers (111, 112) are in parallel with each other, and have a gap therebetween;
wherein the two convoy belts (121, 122) are configured to pass through the gap after they are loaded with the content 140 on their sampling surfaces (121s, 122s), and to carry the loaded content 140 to the sample depository 141;
wherein the contents 140 loaded on the two convoy belts (121, 122) are combined, and the combined contents 140 are sandwiched between the two convoy belts (121, 122); and
wherein a manual or powered collector driver 150 is configured to pull the two convoy belts (121, 122) loaded with the combined contents 140 at a same speed toward the sample depository 141.

7. The catheter-shaped sampling device according to claim 6, wherein the first belt source 131 comprises a roll of the first convoy belt 121 around a roller, the second belt source 132 comprises a roll of the second convoy belt 122 around a roller, and the sample depository 141 comprises a collection spool or reel around which the combined contents 140 sandwiched between the two convoy belts (121, 122) can coil or wrap.

8. The catheter-shaped sampling device according to claim 6, wherein the collector driver 150 comprises a motor that can rotate the collection spool or reel and pull the combined contents 140 sandwiched between the two convoy belts (121, 122) toward the collection spool or reel.

9. The catheter-shaped sampling device according to claim 6, wherein the convoy belt (121 or 122) each comprises a substrate layer and an adsorbent layer having a sampling surface, and wherein the adsorbent layer is continuous, homogenous, flat, smooth and free of any indentations and pockets.

10. The catheter-shaped sampling device according to claim 9, wherein adsorbent layer comprises a material selected form sodium carboxymethylcellulose, polyacrylamide, polyacrylonitrile and acrylic acid polymers, cross-linked acrylic, Karaya gum and polysaccharides.

11. The catheter-shaped sampling device according to claim 1, wherein the biological conduit is selected from a human conduit; an animal conduit; intestinal tract or gut; gastrointestinal tract; buccal cavity; pharynx; esophagus; stomach; small intestine; duodenum; jejunum; ileum; large intestine; cecum; colon; rectum; anal canal; respiratory tract; upper respiratory tract; lower respiratory tract; nasal cavity; paranasal sinuses; pharynx; nasopharynx, oropharynx; laryngopharynx; larynx; trachea; primary bronchi; secondary bronchi; tertiary bronchi; bronchioles (including terminal and respiratory); lungs (including alveoli); an ear canal; vagina; cervix; uterus or womb; Fallopian tubes; ovaries; urinary tract; kidney; renal pelvis; ureter; urinary bladder; and urethra.

12. A process for sampling a content in a biological conduit using the catheter-shaped sampling device according to claim 1, comprising
inserting the flexible tube 11 into the conduit;
moving the flexible tube 11 along the conduit; and
collecting the content of the conduit at a targeted location with a discontinuous sample collector 14 and/or a continuous sample collector.

12. The process according to claim 12, wherein the discontinuous sample collector 14 includes
a sampling wire 1401 within the sampling channel 13,
sampling hairs 1402 extended from a distal and terminal portion of the sampling, wire 1401, and
a protective cap 1403 connected to a distal tip of the sampling wire 1401;
wherein the protective cap 1403 is slightly bigger than the distal opening 130 such that the protective cap 1403 can cover and seal the distal opening 130 to prevent the sampling hairs 1402 and/or samples 16 adhered to the sampling hairs 1402 within the sampling channel 13 from contamination by foreign substances.

14. The process according to claim 13, wherein the step of collecting the content of the conduit includes
pushing the sampling hairs 1402 out from the sampling channel 13,
adhering a sample to the sampling hairs 1402,
pulling the sampling hairs 1402 with the adhered sample back into the sampling channel 13, and
sealing the distal opening 130 of the sampling channel 13 with the protective cap 1403.

15. The process according to claim 13, further comprising
providing a convoy belt, and
continuously loading the sample content collected from the conduit onto the convoy belt.

16. The process according to claim 15, which is implemented with a continuous sample collector 24 that includes
a first cylindrical roller 111;
a first convoy belt 121 having a first sampling surface 121s and a first back surface 121b;
a first belt source 131 for providing the first convoy belt 121 which is blank;
a sample depository 141, wherein the first convoy belt 121 is configured to extend from the first belt source 131 to the first cylindrical roller 111, to contact the roller 111 with the first back surface 121b, to wind around the roller 111, to expose the first sampling surface 121s to the content 140 in the conduit, to load a portion of the content 140 on the first sampling surface 121s, and to carry the loaded content 140 to the sample depository 141;
a second cylindrical roller 112;
a second convoy belt 122 having a second sampling surface 122s and a second back surface 122b; and
a second belt source 132 for providing the second convoy belt 122 which is blank;
wherein the second convoy belt 122 is configured to extend from the second belt source 132 to the second cylindrical roller 112, to contact the roller 112 with the second back surface 122b, to wind around the second cylindrical roller 112, to expose the second sampling surface 122s to the content 140 in the conduit, to load the content 140 on the second sampling surface 122s, and to carry the loaded content 140 to the sample depository 141;
wherein the two cylindrical rollers (111, 112) are in parallel with each other, and have a gap therebetween;
wherein the two convoy belts (121, 122) are configured to pass through the gap after they are loaded with the content 140 on their sampling surfaces (121s, 122s), and to carry the loaded content 140 to the sample depository 141;
wherein the contents 140 loaded on the two convoy belts (121, 122) are combined, and the combined contents 140 are sandwiched between the two convoy belts (121, 122); and
wherein a manual or powered collector driver 150 is configured to pull the two convoy belts (121, 122) loaded with the combined contents 140 at a same speed toward the sample depository 141.

17. The process according to claim 16, wherein the convoy belt (121 or 122) comprises a substrate layer and an adsorbent layer having a sampling surface, and wherein the adsorbent layer is continuous, homogenous, flat, smooth and free of any indentations and pockets.

18. The process according to claim 16, further comprising cutting the loaded convoy belts (121, 122) into pieces for analysis of the sample content 140 collected from the conduit.

19. The process according to claim 12, wherein the catheter-shaped sampling device further comprises other components 15 such as a camera and a lighting device at the distal end 1102 of the flexible tube 11.

20. The process according to claim 12, wherein the biological conduit is selected from a human conduit; an animal conduit; intestinal tract or gut; gastrointestinal tract; buccal cavity; pharynx; esophagus; stomach; small intestine; duodenum; jejunum; ileum; large intestine; cecum; colon; rectum; anal canal; respiratory tract; upper respiratory tract; lower respiratory tract; nasal cavity; paranasal sinuses; pharynx; nasopharynx; oropharynx, laryngopharynx; larynx; trachea; primary bronchi; secondary bronchi; tertiary bronchi; bronchioles (including terminal and respiratory); lungs (including alveoli); an ear canal; vagina; cervix; uterus or womb; Fallopian tubes; ovaries; urinary tract; kidney; renal pelvis; ureter; urinary bladder; and urethra.
